(19) United States
(12) Patent Application Publication
(10) Pub. No.: US 2022/0175626 A1
(43) Pub. Date: Jun. 9, 2022

(54) Temporary Hair Coloring

(71) Applicant: Xerox Corporation, Norwalk, CT (US)

(72) Inventors: Yu Qi, Penfield, NY (US); Judith Millner Vandewinckel, Livonia, NY (US); Eliud Robles Flores, Rochester, NY (US)

(73) Assignee: Xerox Corporation

(21) Appl. No.: 17/114,769

(22) Filed: Dec. 8, 2020

(51) Int. Cl. A61K 8/02; A61K 8/06; A61K 8/87; A61K 8/85; A61K 8/81; A61K 8/9794; A61K 8/92; A61K 8/49; A61K 8/31; A61K 8/19; A61Q 5/06

(52) U.S. Cl. CPC A61K 8/0245; A61K 8/06; A61K 8/87; A61K 8/85; A61K 8/8117; A61Q 5/065; A61K 8/922; A61K 8/492; A61K 8/4926; A61K 8/31; A61K 8/19; A61K 8/9794

(57) ABSTRACT

A temporary hair coloring composition or gloss composition including a core-shell toner particle; wherein the core comprises an optional dye, an optional pigment, an optional additive; and a polymer binder; wherein the shell comprises a shell polymer and an optional additive; and a vehicle comprising a liquid, a paste, or a semi-solid; wherein the vehicle is non-toxic and does not harm hair.

### BACKGROUND

Disclosed herein is a temporary hair coloring composition or gloss treatment composition comprising: a core-shell toner particle; wherein the core comprises an optional dye, an optional pigment, an optional additive; and a polymer binder; wherein the shell comprises a shell polymer and an optional additive; and a vehicle comprising a liquid, a paste, or a semi-solid; wherein the vehicle is non-toxic and does not harm hair.

Also disclosed herein is a temporary hair color kit comprising: separately, at least one core-shell toner particle; wherein the core comprises a dye, a pigment, an optional additive; and a polymer binder; and wherein the shell comprises a shell polymer and an optional additive; optionally, separately, a clear core-shell toner particle; and separately, at least one vehicle comprising a liquid, a paste, or a semi-solid; wherein the vehicle is non-toxic and does not harm hair; wherein one or more temporary hair color compositions is prepared by selecting one or more of the core-shell toner particles and combining with one or more of the vehicles; and wherein one or more clear hair treatments is prepared by selecting the clear core-shell toner particle and combining with one or more of the vehicles.

Also disclosed herein is a process comprising: providing a core-shell toner particle; wherein the core comprises an optional dye, an optional pigment, an optional additive; and a polymer binder; wherein the shell comprises a shell polymer and an optional additive; and combining the core-shell toner particle with a vehicle comprising a liquid, a paste, or a semi-solid; wherein the vehicle is non-toxic and does not harm hair; to form a temporary hair coloring composition when the optional dye or pigment is present; or to form a gloss treatment when the optional pigment or optional dye is not present.

Temporary hair color can be creative and useful for many occasions. Specialty colors are desirable for events, parties, performances, appearances for entertaining, and other purposes.

Temporary hair color is applied to the hair surface and, as contrasted with permanent hair color, can be readily removed, typically with one shampoo. Desirable attributes for temporary hair color include that the color be washable, easy to apply, and inexpensive.

One temporary hair dye composition is described in U.S. Patent Publication 2004/0055094, which is hereby incorporated by reference herein in its entirety. U.S. Patent Publication 2004/0055094 describes a temporary hair dye based on a complex formed by a combination of cationic dye materials with water-soluble anionic polymers. This dye complex is dispersed in a cosmetic vehicle.

While currently available temporary hair coloring compositions may suitable for their intended purposes, a need remains for improved temporary hair coloring compositions. Further, a need remains for temporary hair coloring compositions that are washable, easy to apply, and inexpensive. Further, a need remains for temporary hair coloring compositions that can be prepared at home and applied to wet or dry hair. Further, a need remains for temporary hair coloring compositions that can be applied to produce an ombre effect, for dying the ends of long hair, for producing a high light color, a specialty color, for roots touch-up, and for coloring facial hair. Further, a need remains for temporary hair coloring compositions that are not harmful to hair, that are simple to prepare, easy to use, inexpensive, and that can be removed by washing, such as with a single shampooing.

The appropriate components and process aspects of the each of the foregoing U.S. patents and patent Publications may be selected for the present disclosure in embodiments thereof. Further, throughout this application, various publications, patents, and published patent applications are referred to by an identifying citation. The disclosures of the publications, patents, and published patent applications referenced in this application are hereby incorporated by reference into the present disclosure to more fully describe the state of the art to which this invention pertains.

### SUMMARY

Described is a temporary hair coloring composition comprising: a core-shell toner particle; wherein the core comprises an optional dye, an optional pigment, an optional additive; and a polymer binder; wherein the shell comprises a shell polymer and an optional additive; and a vehicle comprising a liquid, a paste, or a semi-solid; wherein the vehicle is non-toxic and does not harm hair.

Also described is a temporary hair color kit comprising: separately, at least one core-shell toner particle; wherein the core comprises a dye, a pigment, an optional additive; and a polymer binder; and wherein the shell comprises a shell polymer and an optional additive; optionally, separately, a clear core-shell toner particle; and separately, at least one vehicle comprising a liquid, a paste, or a semi-solid; wherein the vehicle is non-toxic and does not harm hair; wherein one or more temporary hair color compositions is prepared by selecting one or more of the core-shell toner particles and combining with one or more of the vehicles; and wherein one or more clear hair treatments is prepared by selecting the clear core-shell toner particle and combining with one or more of the vehicles.

A process comprising: providing a core-shell toner particle; wherein the core comprises an optional dye, an optional pigment, an optional additive; and a polymer binder; wherein the shell comprises a shell polymer and an optional additive; and combining the core-shell toner particle with a vehicle comprising a liquid, a paste, or a semi-solid; wherein the vehicle is non-toxic and does not harm hair; to form a temporary hair coloring composition when the optional dye or pigment is present; or to form a gloss treatment when the optional pigment or optional dye is not present.

### BRIEF DESCRIPTION OF THE DRAWINGS

FIG. 1 is an illustration of hair that has been colored in accordance with the present temporary hair coloring composition to produce an ombre effect.

FIG. 2 is an illustration of hair that has been colored in accordance with the present temporary hair coloring composition to produce various highlights.

FIG. 3 is an illustration of hair that has been colored in accordance with the present temporary hair coloring composition to produce various a metallic effect.

FIG. 4 is an illustration of hair that has been colored in accordance with the present temporary hair coloring composition using a fluorescent yellow core-shell toner particle colorant to produce. The left image illustrates the effect as it appears under inside light (no UV). The middle image illustrates the effect as it appears under outside light on a cloudy day. The right image illustrates the image as it appears inside under UV light.

### DETAILED DESCRIPTION

A temporary, washable hair coloring is provided that can be used for hair, beard, and eyebrows. The hair coloring can be prepared by mixing colorant particles with a media (or vehicle), where the colorant particles are core-shell toner particles, in embodiments, emulsion aggregation core-shell toner particles, and the media is a liquid, paste, or semi-solid. The colorant particles can be one color or can comprise a mixture of process colors. The colorant particles can also comprise a specialty color, for example, a metallic, fluorescent, pearlescent, or phosphorescent color.

The core-shell toner particles that make up the colorant can comprise a core including dyes, pigments, additives, such as antioxidants, UV blockers, and other known toner core additives and a polymer shell to protect the ingredients in the core and prevent the core ingredients from leaking out.

The media can be selected from any suitable or desired vehicle including natural products such as aloe vera gel, shea butter, coconut oil, detergent water, wax, or mixtures thereof.

The temporary hair color does not harm hair and is easy to remove, such a with a single shampooing. The temporary hair color is easy to prepared even at home and is easy to apply to hair. The temporary hair color can be used for any suitable or desired application including for providing a temporary ombre effect, for dying the ends of long hair, for providing high light color, specialty color, roots touch-up, and for coloring facial hair.

In embodiments, a temporary hair coloring composition herein comprises a colorant comprising a core-shell toner particle; wherein the core comprises a dye, a pigment, an optional additive, and a polymer binder; wherein the shell comprises a shell polymer; and a vehicle comprising a liquid, a paste, or a semi-solid; wherein the vehicle is non-toxic and does not harm hair. The colorant comprising the core-shell toner particle is mixed into the vehicle.

Non-toxic as used herein can mean non-poisonous, non-irritating, generally safe for external use. Does not harm hair as used herein means does not significantly dry hair or cause lasting adverse effects to the hair.

The core-shell toner particle that makes up the colorant portion of the temporary hair coloring composition can be prepared by any suitable or desired method. In certain embodiments, the core-shell toner particles are formed by emulsion aggregation methods where the polymer binder, dye, pigment, optional additives, and other components of the toner are placed in one or more surfactants, an emulsion is formed, toner particles are aggregated, coalesced, optionally washed and dried, and recovered. Thus, in embodiments, the core-shell toner particle herein is an emulsion aggregation core-shell toner particle.

In embodiments, the core-shell toner particle core comprises a polymer binder selected from the group consisting of polyester, polystyrene, polystyrene, acrylate, polystyrene acrylate copolymer, polyurethane, and combinations thereof.

In embodiments, the core-shell toner particle shell comprises a shell polymer selected from the group consisting of polyester, polystyrene, acrylate, polystyrene acrylate copolymer, polyurethane, and combinations thereof. In embodiments, the core-shell particle shell comprises as least one amorphous polyester.

The colorant comprising the core-shell toner particle can include polyester, including crystalline polyester, amorphous polyester, and combinations thereof. In embodiments, the core binder comprises an amorphous polyester, a crystalline polyester, or a combination thereof and the shell comprises at least one amorphous polyester or styrene-acrylate copolymer. In embodiments, the shell is free of crystalline polyester.

Crystalline Resin.

The polymer binder, in embodiments, specifically the polyester core binder, may be a crystalline resin formed by reacting a diol with a diacid in the presence of an optional catalyst. For forming a crystalline polyester, suitable organic diols include aliphatic diols with from about 2 to about 36 carbon atoms, such as 1,2-ethanediol, 1,3-propanediol, 1,4-butanediol, 1,5-pentanediol, 2,2-dimethylpropane-1,3-diol, 1,6-hexanediol, 1,7-heptanediol, 1,8-octanediol, 1,9-nonanediol, 1,10-decanediol, 1,12-dodecanediol, combinations thereof, and the like, including their structural isomers. The aliphatic diol may be, for example, selected in an amount of from about 40 to about 60 mole percent of the resin, from about 42 to about 55 mole percent of the resin, or from about 45 to about 53 mole percent of the resin, and a second diol may be selected in an amount of from about 0 to about 10 mole percent of the resin or from about 1 to 4 mole percent of the resin.

Examples of organic diacids or diesters including vinyl diacids or vinyl diesters selected for the preparation of crystalline resins include oxalic acid, succinic acid, glutaric acid, adipic acid, suberic acid, azelaic acid, sebacic acid, fumaric acid, dimethyl fumarate, dimethyl itaconate, cis-1,4-diacetoxy-2-butene, diethyl fumarate, diethyl maleate, phthalic acid, isophthalic acid, terephthalic acid, naphthalene-2,6-dicarboxylic acid, aphthalene-2,7-dicarboxylic acid, cyclohexane dicarboxylic acid, malonic acid and mesaconic acid, a diester or anhydride thereof. The organic diacid may be selected in an amount of, for example, from about 40 to about 60 mole percent of the resin, from about 42 to about 52 mole percent of the resin, or from about 45 to about 50 mole percent of the resin, and a second diacid can be selected in an amount of from about 0 to about 10 mole percent of the resin.

Polycondensation catalysts which may be utilized in forming crystalline (as well as amorphous) polyesters include tetraalkyl titanates, dialkyltin oxides such as dibutyltin oxide, tetraalkyltins such as dibutyltin dilaurate, and dialkyltin oxide hydroxides such as butyltin oxide hydroxide, aluminum alkoxides, alkyl zinc, dialkyl zinc, zinc oxide, stannous oxide, or combinations thereof. Such catalysts may be utilized in amounts of, for example, from about 0.01 mole percent to about 5 mole percent based on the starting diacid or diester used to generate the polyester resin.

Examples of crystalline resins include polyesters, polyamides, polyimides, polyolefins, polyethylene, polybutylene, polyisobutyrate, ethylene-propylene copolymers, ethylene-vinyl acetate copolymers, polypropylene, mixtures thereof, and the like. Specific crystalline resins may be polyester based, such as poly(ethylene-adipate), poly(propylene-adipate), poly(butylene-adipate), poly(pentylene-adipate), poly(hexylene-adipate), poly(octylene-adipate), poly(ethylene-succinate), poly(propylene-succinate), poly(butylene-succinate), poly(pentylene-succinate), poly(hex-ylene-succinate), poly(octylene-succinate), poly(ethylene-sebacate), poly(propylene-sebacate), poly(butylene-sebacate), poly(pentylene-sebacate), poly(hexylene-sebacate), poly(octylene-sebacate), poly(decylene-sebacate), poly(decylene-decanoate), poly(ethylene-decanoate), poly(ethylene dodecanoate), poly(nonylene-sebacate), poly(nonylene-decanoate), copoly(ethylene-fumarate)-copoly(ethylene-sebacate), copoly(ethylene-fumarate)-copoly(ethylene-decanoate), copoly(ethylene-fumarate)-copoly(ethylene-dodecanoate), copoly(2,2-dimethylpropane-1,3-diol-decanoate)-copoly(nonylene-decanoate), poly(octylene-adipate), and mixtures thereof. Examples of polyamides include poly(ethylene-adipamide), poly(propylene-adipamide), poly(butylene-adipamide), poly(pentylene-adipamide), poly(hexylene-adipamide), poly(octylene-adipamide), poly(ethylene-succinimide), poly(propylene-sebecamide), and mixtures thereof. Examples of polyimides include poly(ethylene-adipimide), poly(propylene-adipimide), poly(butylene-adipimide), poly(pentylene-adipimide), poly(hexylene-adipimide), poly(octylene-adipimide), poly(ethylene-succinimide), poly(propylene-succinimide), poly(butylene-succinimide), and mixtures thereof.

In embodiments, the crystalline polyester is of the formula

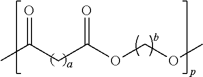

wherein each of a and b may range from 1 to 12, from 2 to 12, or from 4 to 12, and further wherein p may range from 10 to 100, from 20 to 80, or from 30 to 60. In embodiments, the crystalline polyester is poly(1,6-hexylene-1,12-dodecanoate), which may be generated by the reaction of dodecanedioc acid and 1,6-hexanediol.

The designation, “CX:CY,” “CX:Y,” “X:Y,” and forms thereof as used herein describe crystalline resins, wherein C is carbon, X is a positive, non-zero integer identifying the number of methylene groups of the acid/ester monomer used to produce the crystalline polyester (CPE) and Y is a positive, non-zero integer identifying the number of methylene groups of the alcohol monomer used to produce the CPE. Thus, for example, C10 can represent, for example, a dodecanedioic acid and C6 can represent, for example, a hexanediol. X and Y each is 10 or lower. In embodiments, the sum of X and Y is 16 or lower. In certain embodiments, the sum and X and Y is 14 or lower.

In embodiments, the crystalline polyester is a C10:9 resin comprising polyester made from dodecanedioic acid (C10) and 1,9-nonanediol (C9).

As noted above, the crystalline polyesters may be prepared by a polycondensation process by reacting suitable organic diols and suitable organic diacids in the presence of polycondensation catalysts. A stoichiometric equimolar ratio of organic diol and organic diacid may be utilized, however, in some instances where the boiling point of the organic diol is from about 180° C. to about 230° C., an excess amount of diol, such as ethylene glycol or propylene glycol, of from about 0.2 to 1 mole equivalent, can be utilized and removed during the polycondensation process by distillation. The amount of catalyst utilized may vary, and can be selected in amounts, such as for example, from about 0.01 to about 1 or from about 0.1 to about 0.75 mole percent of the crystalline polyester resin.

The crystalline resin may be present in any suitable or desired amount. In embodiments, the crystalline resin may be present, for example, in an amount of from about 1% to about 85% by weight of the toner, from about 5% to about 50% by weight of the toner, or from about 10% to about 35% by weight of the toner.

The crystalline resin can possess various melting points of, for example, from about 30° C. to about 120° C., from about 50° C. to about 90° C. or from about 60° C. to about 80° C. The crystalline resin may have a number average molecular weight (Mn), as measured by gel permeation chromatography (GPC) of, for example, from about 1,000 to about 50,000, from about 2,000 to about 25,000, or from about 5,000 to about 20,000, and a weight average molecular weight (Mw) of, for example, from about 2,000 to about 100,000, from about 3,000 to about 80,000, or from about 10,000 to about 30,000, as determined by GPC. The molecular weight distribution (Mw/Mn) of the crystalline resin may be, for example, from about 2 to about 6, from about 3 to 15 about 5, or from about 2 to about 4.

Amorphous Resin.

The core polymer binder and the shell polymer binder may independently be an amorphous polyester resin formed by reacting a diol with a diacid in the presence of an optional catalyst. Examples of diacids or diesters including vinyl diacids or vinyl diesters utilized for the preparation of amorphous polyesters and include dicarboxylic acids or diesters such as terephthalic acid, phthalic acid, isophthalic acid, fumaric acid, trimellitic acid, dimethyl fumarate, dimethyl itaconate, cis-1,4-diacetoxy-2-butene, diethyl fumarate, diethyl maleate, maleic acid, succinic acid, itaconic acid, succinic acid, succinic anhydride, dodecylsuccinic acid, dodecylsuccinic anhydride, glutaric acid, glutaric anhydride, adipic acid, pimelic acid, suberic acid, azelaic acid, dodecanediacid, dimethyl terephthalate, diethyl terephthalate, dimethylisophthalate, diethylisophthalate, dimethylphthalate, phthalic anhydride, diethylphthalate, dimethylsuccinate, dimethylfumarate, dimethylmaleate, dimethylglutarate, dimethyladipate, dimethyl dodecylsuccinate, and combinations thereof. The organic diacids or diesters may be present, for example, in an amount from about 40 to about 60 mole percent of the resin, from about 42 to about 52 mole percent of the resin, or from about 45 to about 50 mole percent of the resin.

Examples of diols which may be utilized in generating an amorphous polyester include 1,2-propanediol, 1,3-propanediol, 1,2-butanediol, 1,3-butanediol, 1,4-butanediol, pentanediol, hexanediol, 2,2-dimethylpropanediol, 2,2,3-trimethylhexanediol, heptanediol, dodecanediol, bis (hydroxyethyl)-bisphenol A, bis(2-hydroxypropyl)-bisphenol A, 1,4-cyclohexanedimethanol, 1,3-cyclohexanedimethanol, xylenedimethanol, cyclohexanediol, diethylene glycol, bis(2-hydroxyethyl) oxide, dipropylene glycol, dibutylene, and combinations thereof. The amount of organic diols selected may vary, for example, the organic diols may be present in an amount from about 40 to about 60 mole percent of the resin, from about 42 to about 55 mole percent of the resin, or from about 45 to about 53 mole percent of the resin.

Examples of suitable amorphous resins include polyesters, polyamides, polyimides, polyolefins, polyethylene, polybutylene, polyisobutyrate, ethylene-propylene copolymers, ethylene-vinyl acetate copolymers, olypropylene, and the like, and mixtures thereof.

An unsaturated amorphous polyester resin may be utilized as a resin. Examples of such resins include those disclosed in U.S. Pat. No. 6,063,827, the disclosure of which is hereby incorporated by reference in its entirety. Exemplary unsaturated amorphous polyester resins include, but are not limited to, poly(propoxylated bisphenol co-fumarate), poly(ethoxylated bisphenol co-fumarate), poly(butyloxylated bisphenol co-fumarate), poly(co-propoxylated bisphenol co-ethoxylated bisphenol co-fumarate), poly(1,2-propylene fumarate), poly(propoxylated bisphenol co-maleate), poly(ethoxylated bisphenol co-maleate), poly(butyloxylated bisphenol co-maleate), poly(co-propoxylated bisphenol co-ethoxylated bisphenol co-maleate), poly(1,2-propylene maleate), poly(propoxylated bisphenol co-itaconate), poly(ethoxylated bisphenol co-itaconate), poly(butyloxylated bisphenol co-itaconate), poly(co-propoxylated bisphenol co-ethoxylated bisphenol co-itaconate), poly(1,2-propylene itaconate), and combinations thereof.

A suitable polyester resin may be an amorphous polyester such as a poly(propoxylated bisphenol A co-fumarate) resin. Examples of such resins and processes for their production include those disclosed in U.S. Pat. No. 6,063,827, the disclosure of which is hereby incorporated by reference in its entirety.

Suitable polyester resins include amorphous acidic polyester resins. An amorphous acid polyester resin may be based on any combination of propoxylated bisphenol A, ethoxylated bisphenol A, terephthalic acid, fumaric acid, and dodecenyl succinic anhydride, such as poly(propoxylated bisphenol-co-terephthalate-fumarate-dodecenylsuccinate). Another amorphous acid polyester resin which may be used is poly(propoxylated-ethoxylated bisphenol-co-terephthalate-dodecenylsuccinate-trimellitic anhydride).

An example of a linear propoxylated bisphenol A fumarate resin which may be utilized as a resin is available under the trade name SPAMII from Resana S/A Industrias Quimicas, Sao Paulo Brazil. Other propoxylated bisphenol A fumarate resins that may be utilized and are commercially available include GTUF and FPESL-2 from Kao Corporation, Japan, and EM181635 from Reichhold, Research Triangle Park, N.C., and the like.

An amorphous resin or combination of amorphous resins may be present, for example, in an amount of from about 5% to about 95% by weight of the toner, from about 30% to about 90% by weight of the toner, or from about 35% to about 85% by weight of the toner.

The amorphous resin or combination of amorphous resins may have a glass transition temperature of from about 30° C. to about 80° C., from about 35° C. to about 70° C., or from about 40° C. to about 65° C. The glass transition temperature may be measured using differential scanning calorimetry (DSC). The amorphous resin may have a Mn as measured by GPC of, for example, from about 1,000 to about 50,000, from about 2,000 to about 25,000, or from about 1,000 to about 10,000, and a Mw of, for example, from about 2,000 to about 100,000, from about 5,000 to about 90,000, from about 10,000 to about 90,000, from about 10,000 to about 30,000, or from about 70,000 to about 100,000, as determined by GPC.

One, two, or more resins may be used. Where two or more resins are used, the resins may be in any suitable ratio (e.g., weight ratio) such as for instance, of from about 1% (first resin)/99% (second resin) to about 99% (first resin)/1% (second resin), from about 10% (first resin)/90% (second resin) to about 90% (first resin)/10% (second resin). Where the resins include a combination of amorphous and crystal line resins, the resins may be in a weight ratio of, for example, from about 1% (crystalline resin)/99% (amorphous resin) to about 99% (crystalline resin)/I % (amorphous resin), or from about 10% (crystalline resin)/90% (amorphous resin) to about 90% (crystalline resin)/10% (amorphous resin). In some embodiments, the weight ratio of the resins is from about 80% to about 60% of the amorphous resin and from about 20% to about 40% of the crystalline resin. In such embodiments, the amorphous resin may be a combination of amorphous resins, e.g., a combination of two amorphous resins.

In embodiments, the core comprises a solvent yellow-incorporated amorphous resin as described in U.S. patent application Ser. No. 16/676,971, which is hereby incorporated by reference herein in its entirety. In embodiments, the core comprises a first solvent yellow 160-incorporated amorphous polyester comprising a poly(propoxylated bisphenol-co-terephthalate-fumarate-dodecenylsuccinate) and a second solvent yellow 160-incorporated amorphous polyester comprising a poly(propoxylated-ethoxylated bisphenol-co-terephthalate-dodecenylsuccinate-trimellitic anhydride).

Polystyrene, Acrylate, Polystyrene Acrylate Copolymer.

The colorant comprising the core-shell toner particle may employ a polymer binder selected from a polystyrene, an acrylate, a polystyrene acrylate copolymer, and combinations thereof.

Any suitable or desired styrene-acrylate can be selected for the present toner particles. The styrene-acrylate toner selected may be as described in U.S. Pat. No. 5,462,828, which is hereby incorporated by reference herein in its entirety. In embodiments, the toner includes a styrene/n-butyl acrylate copolymer resin having a number average molecular weight of less than about 5,000, a weight average molecular weight of from about 10,000 to about 40,000, and a molecular weight distribution of greater than 6 and provides excellent gloss and high fix properties at a low fusing temperature.

In embodiments, the toner particle resin herein is prepared by polymerizing styrene and n-butyl acrylate monomers. The styrene-n-butyl acrylate resin can be used for the core, the shell, or a combination thereof. The styrene/n-butyl acrylate copolymer resin may be produced by any suitable or desired polymerization method as known in the art. For example, the resins may be produced by conventional solution polymerization or emulsion polymerization including starve fed emulsion polymerization.

In embodiments, the portion of styrene present in the styrene/n-butyl acrylate copolymer resin in from about 60 to about 95 percent by weight, or from about 70 to about 90 percent by weight, or from about 80 to about 88 percent by weight.

In embodiments, the portion of n-butyl acrylate present in the styrene/n-butyl acrylate copolymer resin in from about 5 to about 40 percent by weight, or from about 10 to about 30 percent by weight, or from about 12 to about 20 percent by weight.

Toner.

In order to form the colorant comprising the present core-shell toner particle, any of the resins described above may be provided as an emulsion(s), e.g., by using a solvent-based phase inversion emulsification process. The emulsions may then be utilized as the raw materials to form the toners, e.g., by using an emulsion aggregation and coalescence (EA) process.

In embodiments, a fluorescent colorant is incorporated into an amorphous polyester latex and then the dyed latex is used to make the toner core. See, for example, U.S. patent application Ser. No. 16/676,971, which is hereby incorporated by reference herein in its entirety.

The colorant comprising the core-shell toner particles may be prepared by any method within the purview of one skilled in the art. Although embodiments relating to toner particle production are described below with respect to emulsion-aggregation processes, any suitable method of preparing toner particles may be used, including chemical processes, such as suspension and encapsulation processes disclosed in U.S. Pat. Nos. 5,290,654 and 5,302,486, the disclosures of each of which are hereby incorporated by reference in their entirety. In embodiments, toner particles may be prepared by aggregation and coalescence processes in which small-size resin particles are aggregated to the appropriate toner particle size and then coalesced to achieve the final toner-particle shape and morphology.

In embodiments, core-shell toner particles may be prepared by emulsion-aggregation processes, such as a process that includes aggregating a mixture of an optional wax and any other desired or required additives, and emulsions including the resins described above, optionally in surfactants as described above, and then coalescing the aggregate mixture. A mixture may be prepared by adding an optional wax or other materials, which may also be optionally in a dispersion(s) including a surfactant, to the emulsion, which may be a mixture of two or more emulsions containing the resin. The pH of the resulting mixture may be adjusted by an acid such as, for example, acetic acid, nitric acid or the like. In embodiments, the pH of the mixture may be adjusted to from about 2 to about 4.5. Additionally, in embodiments, the mixture may be homogenized. If the mixture is homogenized, homogenization may be accomplished by mixing at about 600 to about 4,000 revolutions per minute. Homogenization may be accomplished by any suitable means, including, for example, an IKA ULTRA TURRAX® T50 probe homogenizer.

The core may include an additive. The shell may include an additive. In embodiments, the additive in the core of the core-shell toner particle is selected from the group consisting of an antioxidant, a UV blocker, a UV absorber, and combinations thereof. Additionally, other internal and/or external additives may be added in known amounts for their known functions.

In embodiments, the optional additive is present in one or more of the core or the shell, and wherein the optional additive in the core or shell of the core-shell toner particle is independently selected from the group consisting of an antioxidant, a UV blocker, a UV absorber, and combinations thereof.

Following the preparation of the above mixture, an aggregating agent may be added to the mixture. Any suitable aggregating agent may be utilized to form a toner. Suitable aggregating agents include, for example, aqueous solutions of a divalent cation or a multivalent cation material. The aggregating agent may be, for example, polyaluminum halides such as polyaluminum chloride (PAC), or the corresponding bromide, fluoride, or iodide, polyaluminum silicates such as polyaluminum sulfosilicate (PASS), and water soluble metal salts including aluminum chloride, aluminum nitrite, aluminum sulfate, potassium aluminum sulfate, calcium acetate, calcium chloride, calcium nitrite, calcium oxylate, calcium sulfate, magnesium acetate, magnesium nitrate, magnesium sulfate, zinc acetate, zinc nitrate, zinc sulfate, zinc chloride, zinc bromide, magnesium bromide, copper chloride, copper sulfate, and combinations thereof. In embodiments, the aggregating agent may be added to the mixture at a temperature that is below the glass transition temperature (Tg) of the resin.

The aggregating agent may be added to the mixture utilized to form a toner in an amount of, for example, from about 0.1% to about 8% by weight, in embodiments from about 0.2% to about 5% by weight, in other embodiments from about 0.5% to about 5% by weight, of the resin in the mixture. This provides a sufficient amount of agent for aggregation.

In order to control aggregation and coalescence of the particles, in embodiments the aggregating agent may be metered into the mixture over time. For example, the agent may be metered into the mixture over a period of from about 5 to about 240 minutes, in embodiments from about 30 to about 200 minutes. The addition of the agent may also be done while the mixture is maintained under stirred conditions, in embodiments from about 50 rpm to about 1,000 rpm, in other embodiments from about 100 rpm to about 500 rpm, and at a temperature that is below the glass transition temperature of the resin as discussed above, in embodiments from about 30° C. to about 90° C., in embodiments from about 35° C. to about 70° C.

The particles may be permitted to aggregate until a predetermined desired particle size is obtained. A predetermined desired size refers to the desired particle size to be obtained as determined prior to formation, and the particle size being monitored during the growth process until such particle size is reached. Samples may be taken during the growth process and analyzed, for example with a Coulter Counter, for average particle size. The aggregation thus may proceed by maintaining the elevated temperature, or slowly raising the temperature to, for example, from about 40° C. to about 100° C., and holding the mixture at this temperature for a time from about 0.5 hours to about 6 hours, in embodiments from about hour 1 to about 5 hours, while maintaining stirring, to provide the aggregated particles. Once the predetermined desired particle size is reached, then the growth process is halted. In embodiments, the predetermined desired particle size is within the toner particle size ranges known in the art. In embodiments, the toner particles have an average volume particle diameter of from about 4 to about 30 microns.

The growth and shaping of the particles following addition of the aggregation agent may be accomplished under any suitable conditions. For example, the growth and shaping may be conducted under conditions in which aggregation occurs separate from coalescence. For separate aggregation and coalescence stages, the aggregation process may be conducted under shearing conditions at an elevated temperature, for example of from about 40° C. to about 90° C., in embodiments from about 45° C. to about 80° C., which may be below the glass transition temperature of the resin as discussed above.

The latex particles produced as described above may be added to a colorant to produce the core of the core-shell toner particle. In embodiments the colorant may be in a dispersion. The colorant dispersion may include, for example, submicron colorant particles having a size of, for example, from about 50 to about 500 nanometers in volume average diameter and, in embodiments, of from about 100 to about 400 nanometers in volume average diameter. The colorant particles may be suspended in an aqueous water phase containing an anionic surfactant, a nonionic surfactant, or combinations thereof. Suitable surfactants include any of those surfactants described above. In embodiments, the surfactant may be ionic and may be present in a dispersion in an amount from about 0.1 to about 25 percent by weight of the colorant, and in embodiments from about 1 to about 15 percent by weight of the colorant.

Colorants useful in forming toners in accordance with the present disclosure include pigments, dyes, mixtures of pigments and dyes, mixtures of pigments, mixtures of dyes, and the like. The colorant may be, for example, carbon black, cyan, yellow, magenta, red, orange, brown, green, blue, violet, white, or mixtures thereof. In embodiments, the core of the core-shell toner particles comprises a pigment, a dye, a mixture of pigments, a mixture of dyes, a mixture of pigments and dyes, and combinations thereof.

In embodiments wherein the colorant is a pigment, the pigment may be, for example, carbon black, phthalocyanines, quinacridones or RHODAMINE B™ type, red, green, orange, brown, violet, yellow, fluorescent colorants, and the like.

In embodiments, the colorant may be a fluorescent colorant, a metallic colorant, a pearlescent colorant, a phosphorescent colorant, and combinations thereof. Phosphorescent may also be known as glow in the dark. In embodiments, the core comprises a fluorescent colorant. In embodiments, the core comprises a metallic colorant.

Examples of pearlescent pigments include mica particles, surface coated mica particles, metallic pigment, and combinations thereof.

Examples of phosphorescent pigments include semi-conducting inorganic crystal, in embodiments, europium doped strontium aluminate, copper doped zinc sulfide, calcium sulfide and combinations thereof.

Examples of metallic pigments include aluminum flake pigment, copper flake pigment, iron pigment, zinc particles, silver particle, gold particle, and combinations thereof.

In embodiments, the core comprises a member of the group consisting of carbon black, phthalocyanines, quinacridones, cyan, yellow, magenta, red, orange, brown, green, blue, violet, white, fluorescent colorants, metallic colorants, phosphorescent colorants, and mixtures thereof. In embodiments, the core comprises a fluorescent colorant. In embodiments, the core comprises a metallic colorant.

Exemplary colorants include carbon black like REGAL 330) magnetites; Mobay magnetites including M08029™, M08060™; Columbian magnetites; MAPICO BLACKS™ and surface treated magnetites; Pfizer magnetites including CB4799™, CB5300™, CB5600™, MCX6369™; Bayer magnetites including, BAYFERROX 8600™, 8610™; Northern Pigments magnetites including, NP-604™, NP-608™; Magnox magnetites including TMB-100™, or TMB-104™, HELIOGEN BLUE L6900™, D6840™, D7080™, D7020™, PYLAM OIL BLUE™, PYLAM OIL YELLOW™, PIGMENT BLUE 1™ available from Paul Uhlich and Company, Inc.; PIGMENT VIOLET 1™, PIGMENT RED 48™, LEMON CHROME YELLOW DCC 1026™, E.D. TOLUIDINE REDT™ and BON RED C™ available from Dominion Color Corporation, Ltd., Toronto, Ontario; NOVAPERM YELLOW FGLT™, HOSTAPERM PINK E™ from Hoechst; and CINQUASIA MAGENTA™ available from E.I. DuPont de Nemours and Company. Other colorants include 2,9-dimethyl-substituted quinacridone and anthraquinone dye identified in the Color Index as CI 60710, CI Dispersed Red 15, diazo dye identified in the Color Index as CI 26050, CI Solvent Red 19, copper tetra(octadecyl sulfonamido) phthalocyanine, x-copper phthalocyanine pigment listed in the Color Index as CI 74160, CI Pigment Blue, Anthrathrene Blue identified in the Color Index as CI 69810, Special Blue X-2137, diarylide yellow 3,3-dichlorobenzidene acetoacetanilides, a monoazo pigment identified in the Color Index as CI 12700, CI Solvent Yellow 16, a nitrophenyl amine sulfonamide identified in the Color Index as Foron Yellow SE/GLN, CI Dispersed Yellow 33,2,5-dimethoxy-4-sulfonanilide phenylazo-4′-chloro-2,5-dimethoxy acetoacetanilide, Yellow 180, and Permanent Yellow FGL. Organic soluble dyes having a high purity for the purpose of color gamut which may be utilized include Neopen Yellow 075, Neopen Yellow 159, Neopen Orange 252, Neopen Red 336, Neopen Red 335, Neopen Red 366, Neopen Blue 808, Neopen Black X53, Neopen Black X55, wherein the dyes are selected in various suitable amounts, for example from about 0.5 to about 20 percent by weight of the toner, in embodiments, from about 5 to about 18 weight percent of the toner.

In embodiments, colorant examples include Pigment Blue 15:3 having a Color Index Constitution Number of 74160, Magenta Pigment Red 81:3 having a Color Index Constitution Number of 45160:3, Yellow 17 having a Color Index Constitution Number of 21105, and known dyes such as food dyes, yellow, blue, green, red, magenta dyes, and the like.

In other embodiments, a magenta pigment, Pigment Red 122 (2,9-dimethylquinacridone), Pigment Red 185, Pigment Red 192, Pigment Red 202, Pigment Red 206, Pigment Red 235, Pigment Red 269, combinations thereof, and the like, may be utilized as the colorant.

In embodiments, toners of the present disclosure may have high pigment loadings. As used herein, high pigment loadings include, for example, toners having a colorant in an amount of from about 4 percent by weight of the toner to about 40 percent by weight of the toner, in embodiments from about 5 percent by weight of the toner to about 15 percent by weight of the toner. These high pigment loadings may be important for certain colors such as Magenta, Cyan, Black, PANTONE® Orange, Process Blue, PANTONE® yellow, and the like. (The PANTONE® colors refer to one of the most popular color guides illustrating different colors, wherein each color is associated with a specific formulation of colorants, and is published by PANTONE, Inc., of Moonachie, N.J.) One issue with high pigment loading is that it may reduce the ability of the toner particles to spherodize, that is, to become circular, during the coalescence step, even at a very low pH.

In embodiments, the core can be free of colorant to provide a core-shell toner particle that is clear. Combining the core-shell toner particle that is free of colorant in the core with the vehicle herein results in a temporary gloss treatment composition that can be used in the same manner as the temporary hair coloring compositions herein.

The resulting latex, optionally in a dispersion, and colorant dispersion may be stirred and heated to a temperature of from about 35° C. to about 70° C., in embodiments of from about 40° C. to about 65° C., resulting in toner aggregates of from about 2 microns to about 10 microns in volume average diameter, and in embodiments of from about 5 microns to about 8 microns in volume average diameter.

Optionally, a wax may also be combined with the resin in forming toner particles. When included, the wax may be present in an amount of, for example, from about 1 weight percent to about 25 weight percent of the toner particles, in embodiments from about 5 weight percent to about 20 weight percent of the toner particles.

Waxes that may be selected include waxes having, for example, a weight average molecular weight of from about 500 to about 20,000, in embodiments from about 1,000 to about 10,000. Waxes that may be used include, for example, polyolefins such as polyethylene, polypropylene, and polybutene waxes such as commercially available from Allied Chemical and Petrolite Corporation, for example POLYWAX™ polyethylene waxes from Baker Petrolite, wax emulsions available from Michaelman, Inc. and the Daniels Products Company, EPOLENE N-15™ commercially available from Eastman Chemical Products, Inc., and VISCOL 550-P™, a low weight average molecular weight polypropylene available from Sanyo Kasei K. K.; plant-based waxes, such as carnauba wax, rice wax, candelilla wax, sumacs wax, and jojoba oil; animal-based waxes, such as beeswax; mineral-based waxes and petroleum-based waxes, such as montan wax, ozokerite, ceresin, paraffin wax, microcrystalline wax, and Fischer-Tropsch wax; ester waxes obtained from higher fatty acid and higher alcohol, such as stearyl stearate and behenyl behenate; ester waxes obtained from higher fatty acid and monovalent or multivalent lower alcohol, such as butyl stearate, propyl oleate, glyceride monostearate, glyceride distearate, and pentaerythritol tetra behenate; ester waxes obtained from higher fatty acid and multivalent alcohol multimers, such as diethyleneglycol monostearate, dipropyleneglycol distearate, diglyceryl distearate, and triglyceryl tetrastearate; sorbitan higher fatty acid ester waxes, such as sorbitan monostearate, and cholesterol higher fatty acid ester waxes, such as cholesteryl stearate. Examples of functionalized waxes that may be used include, for example, amines, amides, for example AQUA SUPERSLIP 6550™, SUPERSLIP 6530™ available from Micro Powder Inc., fluorinated waxes, for example POLYFLUO 190™, POLYFLUO 200™, POLYSILK 19™, POLYSILK 14™ available from Micro Powder Inc., mixed fluorinated, amide waxes, for example MICROSPERSION 19™ also available from Micro Powder Inc., imides, esters, quatemary amines, carboxylic acids or acrylic polymer emulsion, for example JONCRYL 74™, 89™, 130™, 537™, and 538™, all available from SC Johnson Wax, and chlorinated polypropylenes and polyethylenes available from Allied Chemical and Petrolite Corporation and SC Johnson wax. Mixtures and combinations of the foregoing waxes may also be used in embodiments. Waxes may be included as, for example, fuser roll release agents.

In embodiments, after aggregation, but prior to coalescence, a shell may be applied to the aggregated particles.

Resins which may be utilized to form the shell include, but are not limited to, the amorphous resins described above for use in the core. Such an amorphous resin may be a low molecular weight resin, a high molecular weight resin, or combinations thereof. In embodiments, an amorphous resin which may be used to form a shell in accordance with the present disclosure may include an amorphous polyester of formula I above.

In some embodiments, the amorphous resin utilized to form the shell may be crosslinked. For example, crosslinking may be achieved by combining an amorphous resin with a crosslinker, sometimes referred to herein, in embodiments, as an initiator. Examples of suitable crosslinkers include, but are not limited to, for example free radical or thermal initiators such as organic peroxides and azo compounds described above as suitable for forming a gel in the core. Examples of suitable organic peroxides include diacyl peroxides such as, for example, decanoyl peroxide, lauryl peroxide and benzoyl peroxide, ketone peroxides such as, for example, cyclohexanone peroxide and methyl ethyl ketone, alkyl peroxyesters such as, for example, t-butyl peroxy neodecanoate, 2,5-dimethyl 2,5-di(2-ethyl hexanoyl peroxy) hexane, t-amyl peroxy 2-ethyl hexanoate, t-butyl peroxy 2-ethyl hexanoate, t-butyl peroxy acetate, t-amyl peroxy acetate, t-butyl peroxy benzoate, t-amyl peroxy benzoate, oo-t-butyl o-isopropyl mono peroxy carbonate, 2,5-dimethyl 2,5-di(benzoyl peroxy) hexane, oo-t-butyl o-(2-ethyl hexyl) mono peroxy carbonate, and oo-t-amyl o-(2-ethyl hexyl) mono peroxy carbonate, alkyl peroxides such as, for example, dicumyl peroxide, 2,5-dimethyl 2,5-di(t-butyl peroxy) hexane, t-butyl cumyl peroxide, α-α-bis(t-butyl peroxy) diisopropyl benzene, di-t-butyl peroxide and 2,5-dimethyl 2,5di(t-butyl peroxy) hexyne-3, alkyl hydroperoxides such as, for example, 2,5-dihydro peroxy 2,5-dimethyl hexane, cumene hydroperoxide, t-butyl hydroperoxide and t-amyl hydroperoxide, and alkyl peroxyketals such as, for example, n-butyl 4,4-di(t-butyl peroxy) valerate, 1,1-di(t-butyl peroxy) 3,3,5-trimethyl cyclohexane, 1,1-di(t-butyl peroxy) cyclohexane, 1,1-di(t-amyl peroxy) cyclohexane, 2,2-di(t-butyl peroxy) butane, ethyl 3,3-di(t-butyl peroxy) butyrate and ethyl 3,3-di(t-amyl peroxy) butyrate, and combinations thereof. Examples of suitable azo compounds include 2,2,′-azobis(2,4-dimethylpentane nitrile), azobis-isobutyronitrile, 2,2,-azobis (isobutyronitrile), 2,2,-azobis(2,4-dimethyl valeronitrile), 2,2′-azobis (methyl butyronitrile), 1,1′-azobis(cyano cyclohexane), other similar known compounds, and combinations thereof.

The crosslinker and amorphous resin may be combined for a sufficient time and at a sufficient temperature to form the crosslinked polyester gel. In embodiments, the crosslinker and amorphous resin may be heated to a temperature of from about 25° C. to about 99° C., in embodiments from about 30° C. to about 95° C., for a period of time from about 1 minute to about 10 hours, in embodiments from about 5 minutes to about 5 hours, to form a crosslinked polyester resin or polyester gel suitable for use as a shell.

Where utilized, the crosslinker may be present in an amount of from about 0.001% by weight to about 5% by weight of the resin, in embodiments from about 0.01% by weight to about 1% by weight of the resin. The amount of CCA may be reduced in the presence of crosslinker or initiator.

A single polyester resin may be utilized as the shell or, as noted above, in embodiments a first polyester resin may be combined with other resins to form a shell. Multiple resins may be utilized in any suitable amounts. In embodiments, a first amorphous polyester resin, for example a low molecular weight amorphous resin of formula I above, may be present in an amount of from about 20 percent by weight to about 100 percent by weight of the total shell resin, in embodiments from about 30 percent by weight to about 90 percent by weight of the total shell resin. Thus, in embodiments a second resin, in embodiments a high molecular weight amorphous resin, may be present in the shell resin in an amount of from about 0 percent by weight to about 80 percent by weight of the total shell resin, in embodiments from about 10 percent by weight to about 70 percent by weight of the shell resin.

Following aggregation to the desired particle size and application of the shell, the particles may then be coalesced to the desired final shape, the coalescence being achieved by, for example, heating the mixture to a temperature from about 45° C. to about 100° C., in embodiments from about 55° C. to about 99° C., which may be at or above the glass transition temperature of the resins utilized to form the toner particles, and/or reducing the stirring, for example to from about 100 rpm to about 400 rpm, in embodiments from about 200 rpm to about 300 rpm. The fused particles can be measured for shape factor or circularity, such as with a SYSMEX FPIA 2100 analyzer, until the desired shape is achieved.

Coalescence may be accomplished over a period of time from about 0.01 to about 9 hours, in embodiments from about 0.1 to about 4 hours.

In embodiments, after aggregation and/or coalescence, the pH of the mixture may then be lowered to from about 3.5 to about 6 and, in embodiments, to from about 3.7 to about 5.5 with, for example, an acid, to further coalesce the toner aggregates. Suitable acids include, for example, nitric acid, sulfuric acid, hydrochloric acid, citric acid and/or acetic acid. The amount of acid added may be from about 0.1 to about 30 percent by weight of the mixture, and in embodiments from about 1 to about 20 percent by weight of the mixture.

The mixture may be cooled, washed and dried. Cooling may be at a temperature of from about 20° C. to about 40° C., in embodiments from about 22° C. to about 30° C., over a period of time of from about 1 hour to about 8 hours, in embodiments from about 1.5 hours to about 5 hours.

In embodiments, cooling a coalesced toner slurry may include quenching by adding a cooling media such as, for example, ice, dry ice and the like, to effect rapid cooling to a temperature of from about 20° C. to about 40° C., in embodiments of from about 22° C. to about 30° C. Quenching may be feasible for small quantities of toner, such as, for example, less than about 2 liters, in embodiments from about 0.1 liters to about 1.5 liters. For larger scale processes, such as for example greater than about 10 liters in size, rapid cooling of the toner mixture may not be feasible or practical, neither by the introduction of a cooling medium into the toner mixture, or by the use of jacketed reactor cooling.

Subsequently, the toner slurry may be washed. The washing may be carried out at a pH of from about 7 to about 12, in embodiments at a pH of from about 9 to about 11. The washing may be at a temperature of from about 30° C. to about 70° C., in embodiments from about 40° C. to about 67° C. The washing may include filtering and reslurrying a filter cake including toner particles in deionized water. The filter cake may be washed one or more times by deionized water, or washed by a single deionized water wash at a pH of about 4 wherein the pH of the slurry is adjusted with an acid, and followed optionally by one or more deionized water washes.

Drying may be carried out at a temperature of from about 35° C. to about 75° C., and in embodiments of from about 45° C. to about 60° C. The drying may be continued until the moisture level of the particles is below a set target of about 1% by weight, in embodiments of less than about 0.7% by weight.

The colorant comprising the core-shell toner particle is combined with a media or vehicle to form the temporary hair coloring composition. The vehicle can be any suitable or desired vehicle. In embodiments, the vehicle comprises a member of the group consisting of aloe vera, shea butter, coconut oil, detergent water, wax, and combinations thereof.

The hair coloring composition can be prepared by any suitable or desired method including simple mixing of the core-shell toner particle with the vehicle. In embodiments, a process herein comprises providing a colorant comprising a core-shell toner particle; wherein the core comprises a dye, a pigment, an optional additive; and a polymer binder; wherein the shell comprises a shell polymer; and combining the colorant with a vehicle comprising a liquid, a paste, or a semi-solid; wherein the vehicle is non-toxic and does not harm hair; to form a temporary hair coloring composition.

Combining the core-shell toner particle with the vehicle can include mixing, stirring, blending, homogenizing, beating with an eggbeater, or a combination thereof.

In embodiments, hair coloring kits are provided which include one or more colorants comprising the core-shell toner particles herein and one or more vehicles. The one or more colorants are separately packaged or contained. The one or more vehicles are separately packaged or contained. Thus, a temporary hair color kit here comprises separately, at least one colorant comprising a core-shell toner particle; wherein the core comprises a dye, a pigment, an optional additive; and a polymer binder; and wherein the shell comprises a shell polymer; and separately, at least one vehicle comprising a liquid, a paste, or a semi-solid; wherein the vehicle is non-toxic and does not harm hair; wherein one or more temporary hair color compositions is prepared by selecting one or more of the colorants and combining with one or more of the vehicles.

In embodiments, the temporary hair color kit comprises: separately, at least one colorant comprising a core-shell toner particle; wherein the core comprises a dye, a pigment, an optional additive; and a polymer binder; and wherein the shell comprises a shell polymer; optionally, separately, a clear core-shell toner particle; and separately, at least one vehicle comprising a liquid, a paste, or a semi-solid; wherein the vehicle is non-toxic and does not harm hair; wherein one or more temporary hair color compositions is prepared by selecting one or more of the colorants and combining with one or more of the vehicles; and wherein one or more clear hair treatments is prepared by selecting a clear core-shell toner particle and combining with one or more of the vehicles.

In this embodiment, the kit contains a colorant comprising one ore more separately contained or separately packaged colorants, in embodiments, one or more separately packaged toners as described herein including those selected from the group consisting of black, cyan, yellow, magenta, red, orange, brown, green, blue, violet, white, metallic, fluorescent, pearlescent, and phosphorescent.

The kit may further comprise one or more separately contained or separately packaged vehicles such as vehicles selected from the group consisting of aloe vera, shea butter, coconut oil, detergent water, wax, and combinations thereof.

In this way, an end user can prepare one or more custom colors, for example, at home, to provide a custom color experience. The one or more hair coloring compositions can be applied to the hair using any suitable or desired process, including working the coloring composition into the hair by hand.

The hair coloring composition can be applied to the hair to produce any suitable or desired color or effect such as an ombre effect, highlight colors, specialty colors, metallic colors, high gloss using clear, and other effects depending on the temporary coloring composition selected.

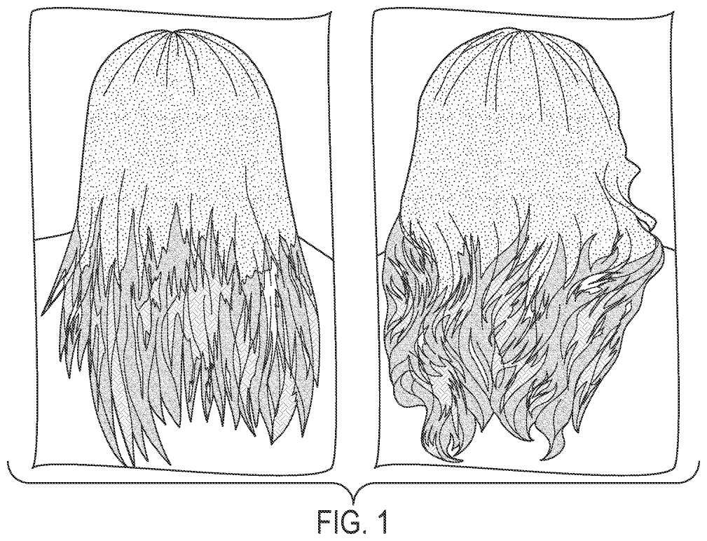

FIG. 1 is an illustration of hair that has been colored in accordance with the present temporary hair coloring composition to produce an ombre effect.

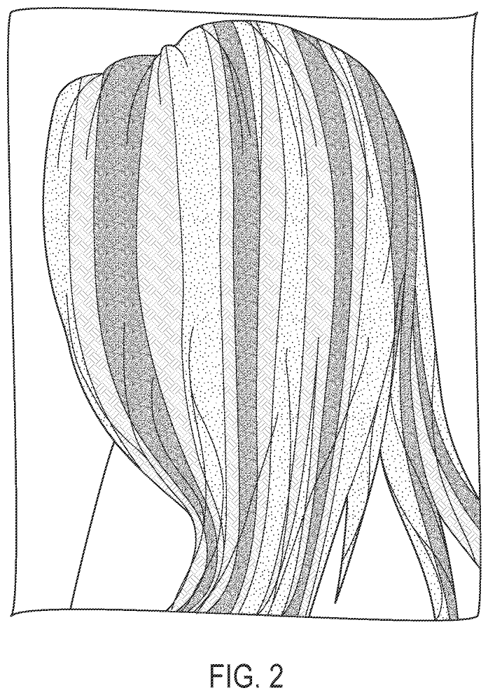

FIG. 2 is an illustration of hair that has been colored in accordance with the present temporary hair coloring composition to produce various highlights.

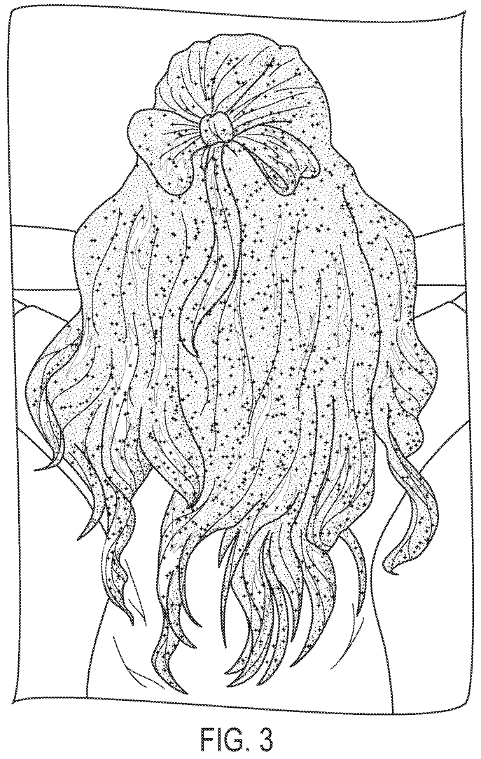

FIG. 3 is an illustration of hair that has been colored in accordance with the present temporary hair coloring composition to produce various a metallic effect.

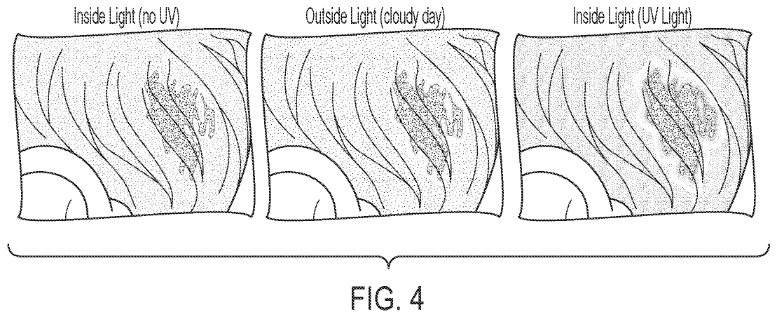

FIG. 4 is an illustration of hair that has been colored in accordance with the present temporary hair coloring composition using a fluorescent yellow core-shell toner particle colorant to produce. The left image illustrates the effect as it appears under inside light (no UV). The middle image illustrates the effect as it appears under outside light on a cloudy day. The right image illustrates the image as it appears inside under UV light.

### EXAMPLES

The following Examples are being submitted to further define various species of the present disclosure. These Examples are intended to be illustrative only and are not intended to limit the scope of the present disclosure.

Also, parts and percentages are by weight unless otherwise indicated.

### Example 1

In a container was placed a mixture of about 5 grams of emulsion aggregation black toner particles and about 100 grams of Aloe Vera (iQ Natural brand). The mixture was stirred using a blender for about 5 to 10 minutes. A homogenizer or eggbeater can also be used. The resulting hair coloring is ready to apply to dry hair.

### Example 2

In a container was placed about 1 gram of fluorescent yellow toner particles. In a separate container was melted about 50 grams of shea butter (Fair Natural brand). The shea butter was slowly poured into the particle container and the resulting mixture was stirred using a blender (or homogenizer or eggbeater) for about 5 to 10 minutes. The resulting hair coloring is ready to apply to dry hair. The hair coloring was manually worked into a swatch of human hair. The hair having the temporary hair coloring applied thereto was uniformly colored and showed bright color under black light.

Thus, a temporary hair coloring composition and process is described providing advantages including: temporary hair coloring comprising core-shell emulsion aggregation particles as colorants and a media; a core comprising dyes, pigments, and optional additives such as antioxidant, UV blocker, and a polymer binder; a shell polymer that is a plural of a polyester mixture selected from various polymers such as polyester, polystyrene, polystyrene acrylate copolymer; media that includes a natural liquid or semi-solid, such a aloe vera gel, shea butter, coconut oil, detergent water; wax, or mixtures thereof; dyes or pigments retained in the core of the particle and protected by a shell, therefore no chemicals leaching out to the scalp or skin; no harm to hair, easy to remove by washing with a single shampoo; simple, easy to use, can be applied to dry hair, and inexpensive; can be used for various applications such as for creating an ombre effect, for dying the ends of hair, for highlight color, for specialty colors using metallic particles, for high gloss with clear toner particles, and other effects.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, shape, angle, color, or material.

1. A temporary hair coloring composition comprising:
a core-shell toner particle;
wherein the core comprises an optional dye, an optional pigment, an optional additive; and a polymer binder;
wherein the shell comprises a shell polymer and an optional additive; and
a vehicle comprising a liquid, a paste, or a semi-solid; wherein the vehicle is non-toxic and does not harm hair.

2. The temporary hair coloring composition of claim 1, wherein the core-shell toner particle is an emulsion aggregation core-shell toner particle.

3. The temporary hair coloring composition of claim 1, wherein the core-shell toner particle core comprises a polymer binder selected from the group consisting of polyester, polystyrene, acrylate, polystyrene acrylate copolymer, polyurethane, and combinations thereof; and
wherein the core-shell toner particle shell comprises a shell polymer selected from the group consisting of polyester, polystyrene, acrylate, polystyrene acrylate copolymer, polyurethane, and combinations thereof.

4. The temporary hair coloring composition of claim 1, wherein the core-shell toner particle shell comprises at least one amorphous polyester or styrene-acrylate copolymer.

5. The temporary hair coloring composition of claim 1, wherein the core comprises a pigment, a dye, a mixture of pigments, a mixture of dyes, a mixture of pigments and dyes, and combinations thereof.

6. The temporary hair coloring composition of claim 1, wherein the core comprises a member of the group consisting of carbon black, phthalocyanines, quinacridones, cyan, yellow, magenta, red, orange, brown, green, blue, violet, white, fluorescent colorants, metallic colorants, pearlescent colorants, phosphorescent colorants, and mixtures thereof.

7. The temporary hair coloring composition of claim 1, wherein the core comprises a fluorescent colorant.

8. The temporary hair coloring composition of claim 1, wherein the core comprises a metallic colorant.

9. The temporary hair coloring composition of claim 1, wherein the vehicle comprises a member of the group consisting of aloe vera, shea butter, coconut oil, detergent water, wax, and combinations thereof.

10. The temporary hair coloring composition of claim 1, wherein the optional additive in the core or shell of the core-shell toner particle is independently selected from the group consisting of an antioxidant, a UV blocker, a UV absorber, and combinations thereof.

11. A temporary hair color kit comprising:
separately, at least one core-shell toner particle; wherein the core comprises a dye, a pigment, an optional additive; and a polymer binder; and wherein the shell comprises a shell polymer and an optional additive;
optionally, separately, a clear core-shell toner particle; and
separately, at least one vehicle comprising a liquid, a paste, or a semi-solid; wherein the vehicle is non-toxic and does not harm hair;
wherein one or more temporary hair color compositions is prepared by selecting one or more of the core-shell toner particles and combining with one or more of the vehicles; and
wherein one or more clear hair treatments is prepared by selecting the clear core-shell toner particle and combining with one or more of the vehicles.

12. The temporary hair color kit of claim 11, wherein the colorant comprises one or more separately contained toner colorants selected from the group consisting of black, cyan, yellow, magenta, red, orange, brown, green, blue, violet, white, metallic, fluorescent, pearlescent, and phosphorescent.

13. The temporary hair color kit of claim 11, wherein the vehicle comprises one or more separately contained vehicles selected from the group consisting of aloe vera, shea butter, coconut oil, detergent water, wax, and combinations thereof.

14. A process comprising:
providing a core-shell toner particle;
wherein the core comprises an optional dye, an optional pigment, an optional additive; and a polymer binder;
wherein the shell comprises a shell polymer and an optional additive; and
combining the core-shell toner particle with a vehicle comprising a liquid, a paste, or a semi-solid; wherein the vehicle is non-toxic and does not harm hair;
to form a temporary hair coloring composition when the optional dye or pigment is present; or
to form a gloss treatment when the optional pigment or optional dye is not present.

15. The process of claim 14, wherein combining comprising mixing, stirring, blending, homogenizing, beating with an eggbeater, or a combination thereof.

16. The process of claim 14, wherein the core-shell toner particle core comprises a polymer binder selected from the group consisting of polyester, polystyrene, acrylate, polystyrene acrylate copolymer, polyurethane, and combinations thereof; and
wherein the core-shell toner particle shell comprises at least one amorphous polyester or styrene-acrylate copolymer.

17. The process of claim 14, wherein the core comprises a member selected from the group consisting of black, cyan, yellow, magenta, red, orange, brown, green, blue, violet, white, metallic colorants, fluorescent colorants, pearlescent colorants, phosphorescent colorants, and combinations thereof.

18. The process of claim 14, wherein the vehicle comprises a member of the group consisting of aloe vera, shea butter, coconut oil, detergent water, wax, and combinations thereof.

19. The process of claim 14, wherein the optional additive in the core of or the shell of the core-shell toner particle is independently selected from the group consisting of an antioxidant, a UV blocker, a UV absorber, and combinations thereof.

20. The process of claim 15, wherein the core-shell toner particle is an emulsion aggregation core-shell toner particle.
